(19) United States
(12) Patent Application Publication
(10) Pub. No.: US 2012/0100204 A1
(43) Pub. Date: Apr. 26, 2012

(54) TGF-Beta Stimulating Agent Compositions and Combinations

(75) Inventor: David J. Grainger, Cambridge (GB)

(73) Assignee: TCP INNOVATIONS LTD, Cambridge (GB)

(21) Appl. No.: 13/280,897

(22) Filed: Oct. 25, 2011

Related U.S. Application Data
(63) Continuation of application No. 11/674,859, filed on Feb. 14, 2007, now abandoned.

(51) Int. Cl. A61K 31/4365; A61P 25/28; A61P 9/00; A61P 37/06; A61K 9/127; A61K 31/60

(52) U.S. Cl. 424/450; 514/301; 514/161

(57) ABSTRACT

Compositions and methods for preventing, inhibiting or treating a disorder associated with the loss of normal adult tissue architecture are provided.

### CLAIM OF PRIORITY

The instant application is a continuation application of U.S. patent application Ser. No. 11/674,859, filed Feb. 14, 2007, the entire content of which is hereby incorporated by reference in its entirety.

### TECHNICAL FIELD

The invention relates to the use of TGF-beta stimulating agents, and in particular members of the triphenylethylene class of pharmaceutical agents, for the prevention, prophylaxis, treatment or amelioration of symptoms of cardiovascular disease, autoimmune diseases or neurodegeneration. In particular, improved compositions consisting of triphenylethylene agents combined with one or more additional active pharmaceutical agents in order to mitigate against side-effects of the triphenylethylene are described and claimed.

### BRIEF DESCRIPTION OF THE FIGURE

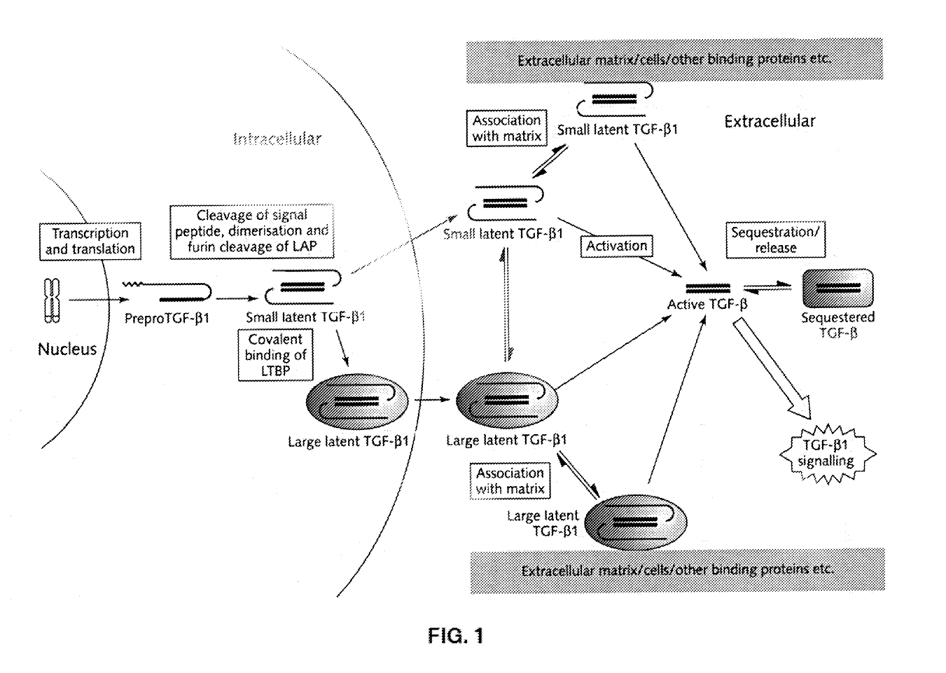

is FIG. 1 shows the pathways involved in the regulation and activation of TGF-beta. The diagram is based on specific data for TGF-beta1, but very similar pathways operate for TGF-beta2 and TGF-beta3. A TGF-beta Production Stimulator, as defined herein, can act on any of these process (or others not illustrated here) in order to increase the amount of local latent TGF-beta available for one or more of the steps marked ‘activation’.

### DESCRIPTION

Many prevalent diseases of middle- and old-age involve the gradual loss of the healthy tissue architecture that was assembled during embryonic and early post-natal development. For example, in coronary artery disease the concentric three-layered structure of the blood vessel wall is disrupted by the gradual development of an atherosclerotic plaque containing cholesterol, smooth muscle cells, calcium, extracellular matrix and cells of the immune system. In autoimmune conditions, the action of antibodies directed against self-antigens mediates a chronic destruction of tissue architecture. Similarly, neurodegenerative conditions such as Alzheimer's Disease result from the deposition of insoluble extracellular matrix protein aggregates and focal recruitment of activated immune cells.

More than a decade ago, we proposed that the maintenance of healthy architecture in a wide range of adult tissues was an active process, and that cytokines in the transforming growth factor type beta (TGF-beta) superfamily were important mediators of this active maintenance (see for example Biochem Soc Trans. 1995 May; 23(2):403-6; Biol Rev Camb Philos Soc. 1995 November; 70(4):571-96). This proposition, termed the Protective Cytokine Hypothesis, was initially controversial, but has subsequently been supported by a wide variety of experimental data (see, for example, Arterioscler. Thromb. Vasc. Biol. 2004 March; 24(3):399-404 and the references therein). For example, when mice are made partially deficient in TGF-beta (whether by heterozygous deletion of the tgfb1 gene or administration of neutralising antibodies or soluble receptors), their susceptibility to atherosclerosis is markedly increased (J Cell Sci. 2000 July; 113(13):2355-61; Arterioscler Thromb. Vasc. Biol. 2002 Jun. 1; 22(6):975-82; Circ Res. 2001 Nov. 9; 89(10):930-4; Blood. 2003 Dec. 1; 102(12):4052-8). Similarly, reduced levels of TGF-beta in genetically modified animals have also been shown to increase pre-disposition to cancer (for example, Nat. Med. 1998 July; 4(7):802-7) and autoimmune diseases (J. Autoimmun. 2000 February; 14(1):23-42).

If reduced levels of TGF-beta predisposes an individual to diseases associated with gradual loss of adult tissue architecture, such as atherosclerosis, autoimmune diseases and neurodegenerative diseases, then agents which increase TGF-beta levels should consequently be protective (see for example Nat. Med. 1996 April; 2(4):381-5; Curr Alzheimer Res. 2005 April; 2(2):183-6).

Unfortunately, however, excessive levels of TGF-beta can be as damaging as reduced levels. Members of the TGF-beta family of cytokines are among the most powerful inducers of extracellular matrix formation known. As a result, if levels of TGF-beta become too high then tissue architecture becomes disrupted through exuberant production of matrix proteins such as collagen or fibronectin, which eventually disrupt the ordered relationship between the cells that compose the tissue (see for example Proc Natl Acad Sci USA. 1993 Nov. 15; 90(22):10759-63 for the effects of excessive TGF-beta on blood vessel wall architecture).

Consequently, it soon became clear that the optimal intervention for the prevention of diseases associated with a loss of adult tissue architecture would be administration of an agent or agents capable of maintaining the level of TGF-beta in the optimal range.

Direct administration of TGF-beta protein is unlikely to fulfil this criterion: like most proteins, TGF-beta shows poor pharmacokinetics (being cleared from the blood within minutes of administration (J Clin Invest. 1991 January; 87(1):39-44)) ensuring that continuous administration would be required to prevent peaks and troughs in the tissue concentration of the protein, taking the level outside of the desired optimal range.

In contrast, stimulation of the cellular production of TGF-beta exploits the natural regulatory systems which prevent (under normal circumstances) an excess activity of this fibrogenic cytokine from building up.

TGF-beta is produced as a latent precursor which has no known biological activity. This precursor consists of a disulphide-linked dimer of the TGF-beta gene product, each monomer of which has undergone proteolytic cleavage between the mature cytokine and the LAP (or Latency-Associated Peptide). However, the dimeric LAP remains non-covalently associated with the mature cytokine, and this complex is unable to bind to conventional TGF-beta receptors. Once released into the extracellular environment (possibly associated, via covalent or non-covalent interactions, with a range of different TGF-beta binding proteins), the latent precursor is subjected to an activation step. A wide range of conditions, at least in vitro, result in a conformational change within the LAP (including application of heat, extremes of pH, chaotropic agents, proteases and specific protein:protein interactions, for example with integrins) that splits apart the non-covalent complex. The process is illustrated in FIG. 1.

This activation process is tightly regulated and serves a number of important functions: (1) it allows TGF-beta to be made by one cell type and then localised into the extracellular matrix at a distant site, where it is subsequently activated to have its effects on the nearby cells; (2) it allows a wider range of factors to dynamically control the levels of TGF-beta activity than would be possible if only gene transcription, translation and excretion were regulated; (3) it allows for feedback control to prevent dangerously high levels of TGF-beta activity building up.

One such positive feedback loop is mediated by the protease inhibitor Plasminogen Activator Inhibitor-1 (PAI-1). The levels of PAI-1 are dramatically regulated at the transcriptional level in most cells by TGF-beta activity, via the conventional TGF-beta cell surface receptors (J Biol. Chem. 1991 Jan. 15; 266(2):1092-100). As a result, as TGF-beta levels rise, so do levels of PAI-1 production. PAI-1 is well known to act as an inhibitor of TGF-beta activation (J. Cell Biol. 1990 August; 111(2):757-6), although the precise molecular mechanism through which the inhibition is mediated remains somewhat controversial. It is likely that PAI-1 acts either to inhibit the action of a protease involved in the intracellular cleavage between the LAP and mature cytokine during the initial production of the latent TGF-beta precursor, or else to inhibit an enzyme (again most likely a protease) which cleaves LAP to release the active cytokine (see Bioessays. 2006 June; 28(6):629-41 for a discussion of these issues).

Since PAI-1 production is stimulated by TGF-beta activity, and itself inhibits TGF-beta activation, this forms a powerful feedback loop which prevents the levels of TGF-beta activity rising too high in a particular tissue. However, since TGF-beta stimulates the production of other protease inhibitors (for example, Tissue-inhibitors of Metalloproteinases; TIMPs) it is likely that multiple parallel feedback loops exist, which together provide ample protection against excess production of the latent precursor.

Unfortunately, direct administration of active TGF-beta protein (either by pharmacologic administration, or by genetic manipulation using altered TGF-beta genes encoding a spontaneously active version of the cytokine) bypasses these regulatory processes, and allows excessive levels of TGF-beta activity to build up. In such studies, rampant tissue fibrosis is usually seen, with rapid destruction of tissue architecture.

In contrast, administration of agents which stimulate production of the latent TGF-beta precursor can increase TGF-beta activity in any tissue where the level is sub-optimal without risking excessive activity and resulting fibrogenesis. For this reason, we postulated that TGF-beta Production Stimulators would be a useful new class of therapeutic agents for the treatment of diseases associated with the loss of the adult tissue architecture, including, but not limited to, cardiovascular diseases, autoimmune diseases, and neurodegenerative diseases (see for example U.S. Pat. No. 7,084,171 issued 1 Aug. 2006; U.S. Pat. No. 6,410,587 issued 25 Jun. 2002).

One such class of TGF-beta Production Stimulators are the triphenylethylene (TPE) derivatives, such as Tamoxifen (TMX). Initially developed as estrogen receptor modulators, the TPEs as a class have diverse pharmacological activities. In addition to binding to the two estrogen receptor proteins (ERα and ERJβ), various TPEs have been reported to act as inhibitors of the ATP-binding Cassette transporter proteins (Biochem Biophys Res Commun. 1997 Jun. 27; 235(3):669-74), the enzyme sterol Δ7,8 isomerase (J Clin Oncol. 1995 December; 13(12):2900-5) and the P-glycoprotein transporter (Biopharm Drug Dispos. 2004 October; 25(7):283-9), as well as acting as antioxidants (Biochem Soc Symp. 1995; 61:209-19). In addition, however, a number of TPEs, and most particularly Tamoxifen, have been reported to stimulate the production of TGF-beta in a wide variety of cell types, both in vitro (Am J Clin Oncol. 1991; 14 Suppl 2:S15-20; Biochem J. 1993 Aug. 15; 294(1):109-12) and in vivo (J Steroid Biochem Mol. Biol. 1993 December; 47(1-6):137-42; Nat. Med. 1995 October; 1(10):1067-73).

It was this activity as a TGF-beta Production Stimulator which led us to claim the use of TPEs, including Tamoxifen, for the prevention of diseases associated with the loss of normal adult tissue architecture, including cardiovascular diseases (such as coronary artery disease and restenosis), as well as autoimmune disorders and neurodegenerative disorders (for example in U.S. Pat. No. 7,084,171 and related patents).

Over the past decade, a wide variety of clinical data has been collected that support our granted claims (for example in U.S. Pat. No. 5,472,985; U.S. Pat. No. 5,595,722; U.S. Pat. No. 5,599,844; U.S. Pat. No. 5,770,609; U.S. Pat. No. 5,773,479; U.S. Pat. No. 5,847,007; U.S. Pat. No. 5,945,456; U.S. Pat. No. 6,117,911; U.S. Pat. No. 6,166,090; U.S. Pat. No. 6,197,789; U.S. Pat. No. 6,251,920; U.S. Pat. No. 6,262,079; U.S. Pat. No. 6,395,494; U.S. Pat. No. 6,410,587 and U.S. Pat. No. 7,084,171 which are each incorporated by reference herein) that TPEs, and Tamoxifen in particular, can be used to prevent these diseases associated with loss of normal adult tissue architecture, and in particular prevent death from myocardial infarction secondary to coronary artery disease. For example, Braithwaite and colleagues presented a meta-analysis of the cardiovascular outcomes of more than 27,000 women treated with Tamoxifen for the prevention of breast cancer, and found a relative risk of 0.67 for death from myocardial infarction among chronic Tamoxifen users (J Gen Intern Med. 2003 November; 18(11):937-47). This translates to a 33% reduction in risk, which, if replicated among higher risk groups such as men, would result in at least 10,000 fewer deaths due to myocardial infarction in the UK alone each year. Similarly, Clarke and colleagues demonstrated that Tamoxifen treatment improved endothelial function, a surrogate marker of atherosclerotic disease burden (Circulation. 2001 Mar. 20; 103(11):1497-502). These results have been summarised in our recent review (Grainger & Schofield, Circulation (2005) 112:3018-24, which is incorporated by reference herein).

Unfortunately, despite such positive demonstration of efficacy in at least one disease associated with loss of normal adult tissue architecture, Tamoxifen has yet to be widely adopted for use outside of the treatment and prevention of ER-positive breast carcinoma (an application which dominantly depends on its alternative pharmacological function as an estrogen receptor modulator).

The reason for this apparent lack of enthusiasm is the burdensome side-effects which accompany the use of Tamoxifen. It is unsurprising that Tamoxifen has a range of effects (some beneficial, others less so) because of the plethora of pharmacologic and molecular interactions reported for it, as well as other members of the TPE class. Few small molecule pharmaceutical agents in use today are genuinely specific for their intended target, and side-effects frequently limit the application of otherwise highly effective medications.

There are a number of generic approaches which can be adopted to limit the impact of side-effects during drug design and development. One approach would be to design or identify entirely new compositions that retain the intended beneficial effects of the original agent, but are more specific and have less diverse molecular interactions and pharmacologic impacts. However, this approach has several major drawbacks: firstly, there is no generally successful method for identifying such compositions, and it may have been difficult, time consuming and costly to identify even the original agent with the side-effects. Secondly, some or all of the side-effects may be a direct or indirect consequence of the same molecular interaction(s) that were responsible for the target beneficial effect. In these instances it will be almost impossible to retain the profile of beneficial effects independently from the side-effects.

A second approach, which has previously been used successfully elsewhere, is to combine more than one active ingredient into a single composition, the combination having superior properties to either component administered alone, or to the same two ingredients administered to the same individual but at different times.

Two different concepts underlie the success of the combination approach: in one scenario two drugs which have similar effects but differing molecular mechanisms of action are combined, such that the two ingredients show a synergistic impact on the target factor. By using two ingredients acting synergistically it is possible to administer markedly lower doses of each ingredient in order to achieve the same beneficial effect. Provided the side-effects do not also show synergistic increases (which, provided they depend on molecular interactions which differ from the target effect, they like will not) such a composition will likely give the same beneficial effects with a reduced burden of side-effects. Indeed, even if the two agents show only additive (as opposed to synergistic) effect then a combined composition will still show reduced side-effects for the same degree of beneficial effect (although the benefit of administering them as a single composition rather than as two separate treatments will likely be less marked). There are numerous examples of such compositions, which combine two active ingredients in a single preparation. For example, Plachetka et al (U.S. Pat. No. 5,872,145 dated Feb. 16, 1999) invented a combination of a 5-HT receptor agonist with an analgesic, particularly an NSAID, for the treatment of migraine. Both active ingredients were administered at a dose below those ordinarily considered as the minimum effective dose for each agent separately, such that the combination together achieved a level of efficacy more commonly associated with administering higher doses of the single agents, each of which is accompanied by unwanted side-effects at doses above the minimum effective dose.

In the second scenario, the second active ingredient in the composition is intended to counter the side-effects of the first active ingredient, so that the combination is simultaneously effective and safe. Such compositions are less common, but patented examples have been very successful in certain applications. For example, the use of estrogen-only hormone replacement therapy leads to undesirable uterine hypertrophy, but the combination of estrogen with a progestogen leads to a combined tablet which can be used safely in women with an in tact uterus, although the unopposed estrogen is equally effective when used in women with hysterectomy (where the side effects cannot manifest themselves). In this example, it is clearly of considerable clinical advantage to combine the two active ingredients in a single composition because the side-effects are sufficiently severe, and may even (in the case of endometrial cancer) be life-threatening, that the single combined composition precludes the possibility of the patient taking one active ingredient without the other.

TPEs such as Tamoxifen have good activity as TGF-beta Production Stimulators, but a number of side-effects have been identified which limit their broader application. Most importantly, chronic use of Tamoxifen at the most commonly used dose (20 mg/day) results in a small but significant increase in thromboembolic events, a proportion of which may be fatal. This increased pro-coagulant tendency among chronic Tamoxifen users may also underlie the increase in fatal cerebrovascular accidents (strokes) among Tamoxifen users (J Gen Intern Med. 2003 November; 18(11):937-47), some 90% of which are ischemic (as opposed to haemorrhagic in origin). These pro-coagulant side-effects are of particular concern in a cardiovascular setting where TPEs were envisioned for the prevention or treatment of coronary artery disease, since the patient may already show pro-coagulant tendencies prior to beginning treatment. Furthermore, patients at increased risk of coronary artery disease are also likely to be at increased risk of ischemic stroke. Other side effects, such as the increased risk of endometrial cancer, may also be of concern, particularly when using TPEs to treat diseases which are prevalent in women, such as autoimmune diseases (e.g. rheumatoid arthritis). More minor side-effects also exist, such as hot flushes and other consequences of the hormonal activity of the TPEs. These more minor side effects significantly affect the quality of life of the patient, and while they would not necessarily preclude the use of these agents for the treatment of severe or life-threatening conditions (including the diseases associated with loss of the normal adult tissue architecture, such as cardiovascular diseases, autoimmune disorders and neurodegeneration), such side-effects cause problems with patient compliance which in turn threatens the effectiveness of such medications even for the treatment of more severe disease.

Here, we describe the first compositions useful as TGF-beta Production Stimulators for the prevention or treatment of diseases associated with the loss of normal adult tissue architecture (including cardiovascular diseases, autoimmune diseases, and neurodegenerative conditions) which reduce or avoid the side-effects which otherwise limit the application of previously described TGF-beta Production Stimulators in these broad indications.

The invention provides the composition and use of a therapeutic agent, comprising at least two active ingredients (as well as any excipient or carrier), where at least one of the active ingredients is a TGF-beta Production Stimulator, and another active ingredient able to reduce the side-effects associated with the administration of the first active ingredient.

More specifically, the invention provides the composition and use of a therapeutic agent, comprising at least two active ingredients, where at least one of the active ingredients is a compound of formula (I), below, and another active ingredient able to reduce or abolish the side-effects associated with the administration of the first active ingredient.

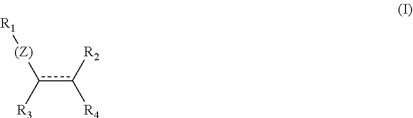

wherein
R₁ is (C₁-C₆)alkyl, or aryl, optionally substituted by 1, 2 or 3 V;
R₂ is phenyl, optionally substituted by 1, 2 or 3 V; or R₂ is (C1-C12)alkyl, halo(C1-C12)alkyl, (C3-C6)cycloalkyl, (C1-C6)alkylcyclo(C3-C6)alkyl, (C5-C6)cycloalkenyl, or (C1-C6)alkyl(C5-C6)cycloalkenyl;
R₃ is hydrogen or phenyl, optionally substituted at the 2-position with R_{J}, and additionally optionally substituted by 1, 2 or 3 V;
R4 is hydrogen, nitro, halo, aryl, heteroaryl, aryl(C1-C3)alkyl, heteroaryl(C1-C3)alkyl, halo(C1-C12)alkyl, cyano(C1-C12)alkyl, (C1-C4)alkoxycarbonyl(C1-C12)alkyl, (C1-C12)alkyl, (C3-C6)cycloalkyl, (C1-C6)alkylcyclo(C3-C6)alkyl, (C5-C6)cycloalkenyl, or (C1-C6)alkyl(C5-C6)cycloalkenyl, wherein any aryl or heteroaryl may optionally be substituted by 1, 2 or 3 V; or
R₅ and Rⱼ together are —CH₂—CH₂—, —S—, —O—(NH)—, —N[(C1-C6)alkyl]-, —OCH₂—, O—C[(C1-C6)alkyl]₂— or —CH═CH—;

— is a single bond or is —C(B)(D)- wherein B and D are each independently hydrogen, (C1-C6)alkyl or halo;
V is OPO₃H₂, (C1-C6)alkyl, (C1-C6)alkoxy, mercapto, (C1-C4)alkylthio, halo, trifluoromethyl, pentafluoroethyl, nitro, N(Rₙ)(Rₒ), cyano, trifluoromethoxy, pentafluoroethoxy, benzoyl, hydroxy, alkyl, benzyl, —OSO₂(CH₂)₀₋₄—CH₃, U(CH₂)₁₋₄COORp, —(CH₂)₀₋₄COORₚ, —U(CH₂)₂₋₄ORₚ, —(CH₂)₀₋₄ORₚ, —U(CH₂)₁₋₄C(═O)Rₖ, —(CH₂)₀₋₄C(═O)Rₖ, —U(CH₂)₁₋₄Rₖ, —(CH₂)₀₋₄Rₖ, or —U(CH₂)₂₋₄OC(═O)Rₚ; wherein U is O, N(Rₘ), or S;
Z is —(CH₂)₁₋₃—, O, —OCH₂—, —CH₂O—, —C(═O)O—, N(R_{q})—, C═O, or a covalent bond;
Rₖ is amino, optionally substituted with one or two (C1-C6)alkyl; or an N-heterocyclic ring optionally containing 1 or 2 additional N(R_{z}), S or nonperoxide O, wherein R_{z} is H, (C1-C6)alkyl, phenyl or benzyl;
Rₙ and Rₒ are independently hydrogen, (C1-C6 alkyl), phenyl, benzyl, or (C1-C6)alkanoyl; or Rₙ and Rₒ together with the nitrogen to which they are attached are a 3, 4, 5 or 6-membered heterocyclic ring;
Rₚ is H or (C1-C6)alkyl; and
Rₘ and R_{q} are independently hydrogen, (C1-C6)alkyl, phenyl, benzyl or (C1-C6)alkanoyl;
or the compound is 1-(4-[2-(diethylamino)ethoxy]phenyl)-2-(4-methoxyphenyl)-1-phenylethan-1-ol (MER25);
   or a pharmaceutically acceptable salt thereof.

Preferably, the compounds of general formula (I) will be a triphenylethylene structure of the formula (II):

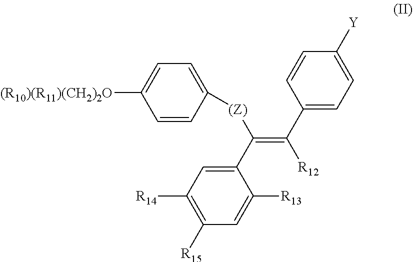

wherein
Z is C═O or a covalent bond;
Y is H or O(C1-C4 alkyl);
R₁₀ and R₁₁ are individually (C1-C4)alkyl or together with the N to which they are bound form a saturated heterocyclic group;
R₁₂ is ethyl or chloroethyl;
R₁₃ is H, or together with R₁₂ is —CH₂—CH₂— or —S—;
R₁₄ and R₁₅ are independently selected among H, I, O(C1-C4)alkyl;
   or a pharmaceutically acceptable salt thereof.

More preferably, the compound (II) is tamoxifen, droloxifene or toremifene.

The second active ingredient in the composition may be selected from one of two categories:

In the first category are compounds which act in a synergistic manner to the first active ingredient (the TGF-beta Production Stimulator), so that the first active ingredient may be administered at a lower dose than would be the case if the first active ingredient were administered alone, and not as part of the combined composition of the invention.

For example, where the composition of the invention is intended to treat or prevent coronary artery disease, suitable active ingredients in this first category would be statins (such as atorvastatin), fibrates (such as fenofibrate), or other lipid-lowering drugs (such as niacin), PPAR agonists (such as roziglitazone), beta blockers (such as atenolol) or ACE inhibitors (such as captopril). In each case, the principle is that co-administration of an agent which also reduces the development or progression of the disease, together with a TGF-beta Production Stimulator administered for the same purpose, allows a lower dose of the TGF-beta Production Stimulator to be administered, thereby reducing or eliminating the side-effects associated with administration of the TGF-beta Production Stimulator, and resulting in an improved risk:benefit profile for the patient compared to administration of either of the two active ingredients singly.

In other words, the combination of two agents which act synergistically allow one or both agents to be administered at lower doses than if either of the two active ingredients were administered alone. The use of lower doses will be associated with reduced side effects for the same level of efficacy.

Preferably, if the second active ingredient is selected from this first category, the second active ingredient will be a statin; more preferably the second active ingredient will be simvastatin or atorvastatin.

Importantly, where the second active ingredient in the combined composition of the invention is selected from this first category, the dose of the first active ingredient in the combined composition must be lower than the optimal dose of that same active ingredient when administered separately, and not as part of the combined composition of the invention. Preferably the first active ingredient will be used in the combined composition at 1-80% of the optimal dose when administered alone; more preferably the dose will be 10-50% of the optimal dose when administered alone.

In the second category of agents suitable for use as the second active ingredient in the combined composition of the invention are compounds which inhibit, reduce or abolish one or more of the specific side-effects due to the inclusion of the first active ingredient, the TGF-beta Production Stimulator.

For example, where the composition of the invention includes a triphenylethylene of structure I as the TGF-beta Production Stimulator, agents with anti-coagulant activity would be selected to reduce or abolish the pro-coagulant side-effects of the triphenylethylene. Agents in the second category therefore include, but are not limited to the following: anti-platelet agents (for example aspirin, aspirinates, clopidogrel, tirofiban, RGD-containing peptides, adenosine and related agents, and prostacyclin and long-lived analogs), oral anticoagulants (for example warfarin, coumarinoids, heparins including low-molecular weight heparin, direct thrombin inhibitors including ximelagatran, and factor Xa inhibitors including hirudin), as well as other agents which have similar anticoagulant activity.

Preferably, if the second active ingredient is selected from the second category, the second active ingredient will be an anti-platelet agent; preferably, the second active ingredient will be a compound of structure (III); preferably the second active ingredient will be aspirin or clopidogrel.

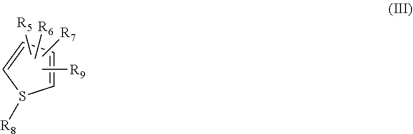

where
R₅ is hydrogen, halo, nitro, cyano, hydroxy, CF₃, —NR_{c}R_{d}, —C(═O)ORₑ, —OC(═O)ORₑ, —C(═N)ORₑ, (C1-C6)alkyl or (C1-C6)alkoxy;
R₆ is hydrogen or —XRₐ;
R₇ is —C(═O)YR_{b};
R₈ is (═O)ₙ; or R₈ is (C1-C6)alkyl, (C1-C6)alkanoyl or (C1-C6)alkanoyloxy and forms a sulfonium salt with the thiophene sulphur, wherein the associated counter ion is a pharmaceutically acceptable anion;
R₉ is hydrogen, —C(═O)ORₕ or —C(═O)SRₕ;
n=0, 1 or 2;
X is oxygen or sulphur;
Y is oxygen or sulphur;
Rₐ is (C1-C6)alkanoyl;
R_{b} is hydrogen or (C1-C3) alkyl;
R_{c} and R_{d} are each independently hydrogen, (C1-C4)alkyl, phenyl, C(═O)OH, C(═O)O(C1-C4)alkyl, CH₂C(═O)OH, CH₂C(═O)O(C1-C4)alkyl, or (C1-C4)alkoxy; or
R_{c} and R_{d} together with the nitrogen to which they are attached are a 3, 4, 5 or 6 membered heterocyclic ring; and
Rₑ-Rᵢ, are independently hydrogen or (C1-C6)alkyl;
   or a pharmaceutically acceptable salt thereof;
   provided that R₆ and R₇ are on adjacent positions of the ring to which they are attached, or are on the 2- and 5-positions of the ring; and further provided that when R₆ is hydrogen R₇ is on the 2- or 5-position of the ring to which it is attached and R₄ is (C1-C4)alkanoyloxy.

Such active ingredients, of structure III, together with aspirin and salicylic acid, are herein referred to as aspirinates. Where aspirinates contain a carboxylate moiety (as in salicylic acid), the definition also includes salt forms (such as sodium aspirinate). Preferably, in compositions according to the present invention, the counterion will be sodium, potassium or copper.

Importantly, where the second active ingredient in the composition of the invention is selected from the second category, the dose is selected so as to be sufficient to reduce or abolish the side effects associated with the use of the first active ingredient. In contrast, to the dose used for agents selected from the first category, agents selected from the second category will typically be used at doses similar to those used when the agent is intended to treat a disease similar to the iatrogenic side effects of the first compound. For example, in a composition of the invention where the first active ingredient is Tamoxifen, then the major side-effects associated with Tamoxifen use is ischemic stroke resulting from the pro-coagulant effects of the drug. In this example, the composition of the invention would therefore include as a second active ingredient an anticoagulant to reduce or abolish the side effect, while allowing the Tamoxifen (as a TGF-beta Production Stimulator) to prevent or treat the disease. Such a composition would therefore include Tamoxifen at a dose which maximally stimulates TGF-beta production (for example, 20 mg per day) as well as an anticoagulant agent such as clopidogrel at a dose typically used to treat diseases associated with a pro-coagulant status (for example, 75 mg per day). For Example, the dose of the second ingredient may be between two and four times the dose of the first ingredient.

It is envisaged that some agents may be members of both categories (for example, the second active ingredient in the composition of the invention may itself provide synergistic benefit in the treatment of the disease being targeted and at the same time act to reduce the side-effects of the first active ingredient).

It is further envisaged that a composition of the invention may be a fixed dose combination of more than two active ingredients, at least one of which is a TGF-beta Production Stimulator. Typically, such a composition will have either two or three active ingredients. Typically, the composition will contain, in addition to the TGF-beta Production Stimulator, either one further active ingredient selected from either the first or second categories above, or else two further active ingredients (where one is selected from each of the two categories, or where both are selected from the same category). Preferably, where the composition contains three active ingredients, the TGF-beta Production Stimulator will be Tamoxifen, the second active ingredient will be aspirin and the third active ingredient will be clopidogrel.

Importantly, the composition of the invention must be administered to the patient as a mixture. The principal advantage of the composition of the invention over the separate administration of the two active ingredients is safety. The side effects of TGF-beta Production Stimulators, such as triphenylethylenes, can be severe and even lethal in certain circumstances. As a result, it represents an unnecessary risk to allow the administration of the two agents separately, when the possibility exists that the patients may (accidentally or deliberately) continue to have administered one of the active ingredients and not the other active ingredient. In such circumstances, the patient may suffer considerable harm: in the event that the second active ingredient was taken from the first category, then the continued administration of a single active ingredient at a dose below its optimal dose would likely result in loss of efficacy of the medication, or else require an increased dose of the single active ingredient so that the individual became at increased risk of significant side effects. In the event that the second active ingredient was taken from the second category, the continued administration of the first active ingredient would lead to an unnecessary exposure to increased risk of side effects.

For example, in the event that the TGF-beta Production Stimulator selected is Tamoxifen, and the second active ingredient selected is clopidogrel, then the composition of the invention has significant benefit to the patient over the administration of either Tamoxifen or clopidogrel alone, as well as over the separate administration of the two substances to the patient. Most particularly, the separate administration of the two substances runs the risk that the patient may (accidentally or deliberately) continue to take the Tamoxifen, and discontinue the clopidogrel. In such circumstances, while the Tamoxifen would continue to ameliorate the symptoms and progression of the disease, the patient would be at increased risk of suffering a stroke as a result of the pro-coagulant effects of the Tamoxifen. Such a risk is higher in individuals where the pro-coagulant effect of Tamoxifen is not being opposed by an anti-coagulant, such as clopidogrel.

In other words, the provision of two pharmaceutical agents as a single medicament (tablet, capsule, gel or other dosage form) offers considerable advantages over the separate administration of the two pharmaceutical agents, when the desirable effect of the two components together is different from the effects of either agent when administered separately. Although the same effect might, in principle, be achievable by administering the compounds at the same time but as separate medicaments (tablets, capsules or gels, for example), nevertheless the risk of achieving a different (and less desirable) effect similar to either compound administered alone is greater than when the two are administered as a single medicament. Where the difference in effect profile between the combination of the two pharmaceutical agents and either one administered alone is significant (such as in the case of potentially lethal side-effects) then such an increased risk becomes unacceptable.

The invention also provides pharmaceutical compositions comprising at least two active ingredients as a mixture, including a compound which is a TGF-beta Production Stimulator, preferably of formula (I), more preferably of formula (II) or a pharmaceutically acceptable salt thereof, together with a compound which reduces the side-effects associated with the administration of the first compound, and at least one pharmaceutically acceptable excipient and/or carrier. For the purposes of this specification, the term ‘mixture’ may optionally include a chemical combination, such as a salt, composed of the two agents according to the invention. Alternatively, the chemical combination may be an ester, or an amide or any similar covalent chemical linkage which allows both components to retain their full pharmaceutical activity. Examples of such compositions, according to the present invention, would be the salt tamoxifen aspirinate (where the Tamoxifen acts as a TGF-beta Production Stimulator, and the aspirinate counter ion is both a synergistic stimulator of TGF-beta production; see Example 1 below) and an anti-platelet agent reducing the pro-coagulant side-effects of the Tamoxifen. It is further envisioned that such ‘dual-action’ salts may be further combined with other agents according to the present invention, such that a composition composed of a mixture of Tamoxifen aspirinate and clopidogrel would fall under the scope of the present invention.

By pharmaceutically acceptable salt is meant in particular the addition salts of inorganic acids such as hydrochloride, hydrobromide, hydroiodide, sulphate, phosphate, diphosphate and nitrate or of organic acids such as acetate, maleate, fumarate, tartrate, succinate, citrate, lactate, methanesulphonate, p-toluenesulphonate, palmoate and stearate. Also within the scope of the present invention, when they can be used, are the salts formed from bases such as sodium or potassium hydroxide. For other examples of pharmaceutically acceptable salts, reference can be made to “Salt selection for basic drugs”, Int. J. Pharm. (1986), 33, 201-217.

The pharmaceutical composition can be in the form of a solid, for example powders, granules, tablets, gelatin capsules, liposomes or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax. Other appropriate pharmaceutically acceptable excipients and/or carriers will be known to those skilled in the art.

The pharmaceutical compositions according to the invention can also be presented in liquid form, for example, solutions, emulsions, suspensions or syrups. Appropriate liquid supports can be, for example, water, organic solvents such as glycerol or glycols, as well as their mixtures, in varying proportions, in water.

In particular, preferred compositions according to the invention are selected from the following list:
Tamoxifen and aspirin, droloxifene and aspirin or toremifene and aspirin;
Tamoxifen and clopidogrel, droloxifene and clopidogrel or toremifene and clopidogrel;
Tamoxifen and aspirin and clopidogrel;
Tamoxifen and atorvastatin or Tamoxifen and simvastatin;
Tamoxifen aspirinate, droloxifene aspirinate or toremifene aspirinate;
Tamoxifen aspirinate and clopidogrel or Tamoxifen aspirinate and atorvastatin;
Tamoxifen and naproxen or Tamoxifen aspirinate and naproxen;
Tamoxifen and galantamine or Tamoxifen aspirinate and galantamine;
   and (except where specific salts are already specified) any pharmaceutically acceptable salts thereof.

The invention includes compounds, compositions and uses thereof as defined, wherein the compound is in hydrated or solvated form.

A preferred composition according to the invention consists of 15 mg Tamoxifen (either as citrate or aspirinate salt) combined with 50 mg of aspirin, or with 50 mg of clopidogrel, or with 50 mg of each of aspirin and clopidogrel, the said composition in tablet form (with appropriate pharmaceutical carriers or excipients). Preferably tablets of such composition would be administered to the patient on two (or more) occasions each day. The principal advantage of splitting the daily dosage in this way is to maintain maximal anti-platelet activity across each 24 hour period (compared to dosing once per day, when, due to the pharmacokinetics of the anti-platelet agents being used, the activity begins to decline prior to the next dose being taken).

According to this invention, disorders intended to be prevented or treated by the compositions of the invention, or the pharmaceutically acceptable salts thereof or pharmaceutical compositions or medicaments containing them as active ingredients include notably:
autoimmune diseases, for example such as multiple sclerosis, rheumatoid arthritis, Crohn's disease, Grave's disease, myasthenia gravis, lupus erythrematosus, scleroderma, Sjogren's syndrome, autoimmune type I diabetes;
vascular disorders including stroke, coronary artery diseases, myocardial infarction, unstable angina pectoris, atherosclerosis or vasculitis, e.g., Behget's syndrome, giant cell arteritis, polymyalgia rheumatica, Wegener's granulomatosis, Churg-Strauss syndrome vasculitis, Henoch-Schönlein purpura and Kawasaki disease;
asthma, allergic rhinitis or chronic occlusive pulmonary disease (COPD);
osteoporosis (low bone mineral density);
tumor growth;
organ transplant rejection and/or delayed graft or organ function, e.g. in renal transplant patients;
psoriasis;
allergies;
Alzheimer's disease, and other idiopathic dementias resulting from neurodegeneration;
Parkinson's disease;
Huntington's disease;
Traumatic brain injury (such as head injuries resulting from a motor vehicle accident), as well as the chronic sequelae (such as impaired memory) resulting from such acute traumatic injuries.

Where legally permissible, the invention also provides a method of treatment, amelioration or prophylaxis of the symptoms of a disease involving the loss of normal adult tissue architecture by the administration to a patient of a therapeutically effective amount of a composition or medicament as claimed herein.

Administration of a medicament according to the invention can be carried out by topical, oral, parenteral route, by intramuscular injection, etc.

The administration dose envisaged for a medicament according to the invention is comprised between 0.1 mg and 10 g depending on the type of active compound used.

Preferably, the diseases ameliorated, treated or prevented by the administration of the compositions of the invention are selected from the following list:
Cardiovascular diseases, including atherosclerosis, and the clinical sequellae of atherosclerosis, such as myocardial infarction, angina pectoris, unstable angina, stroke, transient ischemic attack and peripheral occlusive artery disease;
Autoimmune diseases, including rheumatoid arthritis and multiple sclerosis; and
Neurodegenerative diseases, including Alzheimer's Disease and Parkinson's Disease.

The compositions of the invention are readily manufactured using methods which are well known in the art. In particular, the individual active pharmaceutical ingredients may be synthesised by methods well known in the art, and many are commercially available. Except where the two or more active ingredients are chemically combined, the two or more active pharmaceutical ingredients which compose the composition of the invention are then mixed together, preferably as a finely divided powder so that a homogenous mixture is achieved, then added to appropriate pharmaceutical carriers and/or excipients using techniques well known in the art. The mixture, together with any carriers and excipients, is then prepared in a form suitable for administration to a human, for example as a tablet, capsule, liquid suspension or suppository, using methods well established in the art.

Where the composition of the invention includes two or more active pharmaceutical ingredients which are chemically combined, for example as a salt, then the combination is prepared using methods well known in the art. For example, to prepare a salt such as Tamoxifen aspirinate a solution of Tamoxifen free base in an appropriate solvent (such as DMSO or ethanol) is treated with an equimolar amount of acetylsalicylic acid, the acid and base then react together to form the salt (plus water). After an appropriate period of time (for example, overnight), the solvent is removed, for instance by use of a vacuum pump, and the solid salt can be used as the composition of the invention. Other methods of counterion exchange are well known in the art, and can be similarly be used to prepare Tamoxifen aspirinate from alternative starting materials, such as Tamoxifen citrate and sodium aspirinate.

Where the composition of the invention includes two or more active pharmaceutical ingredients which are chemically combined, in a single covalently linked compound (for example, an ester of 4-hydroxytamoxifen and salicylic acid), the ester is prepared by methods well known in the art. For example, a mixture of 4-hydroxytamoxifen and salicylic acid in an appropriate solvent (such as toluene) may be induced to form an ester by either acid-catalysis or base catalysis depending on the stability of the constituents. Alternatively, an activated form of the acid component can first be prepared (such as an acid chloride or an acid anhydride) which will react with the hydroxylated component directly without the need for catalysis. The general methods for the preparation of such activated acid intermediates, and their subsequent use to form esters are well known in the art.

The following examples are presented in order to illustrate the above procedures and should in no way be considered to limit the scope of the invention.

### Example 1

### Unexpected Synergistic Effects of Tamoxifen and Aspirin in Cell Culture

A preferred composition according to the invention is a mixture composed of Tamoxifen as the first active ingredient (a well known TGF-beta Production Stimulator; see for example U.S. Pat. No. 6,262,079 and U.S. Pat. No. 6,410,587) and aspirin as the second active ingredient, selected to reduce the pro-coagulant side-effects associated with Tamoxifen use.

In order to test the impact of combining the ingredients on the stimulation of TGF-beta production, which is the primary mode of efficacy of the compositions of the invention, we compared the ability of the combined composition to stimulate TGF-beta mRNA and protein production in a cell culture model, and compared the combination with the two agents administered separately.

### Methods

We selected explant-derived human aortic vascular smooth muscle cells (Clonetics Corp) as the target cell type for this experiment, because these cells have previously been shown to stimulate TGF-beta production in response to Tamoxifen (see for example Kirschenlohr et al. (1995) Cardiovasc. Res. 29:848-55). The cells were cultured (37° C.; 5% CO₂) in DMEM+20% foetal calf serum (FCS), and subcultured every 5 days at 1:2 dilution, using 0.02% trypsin/EDTA (Gibco).

For the experiments, the cells were subcultured into 12-well cluster plates at 1×10⁵ cells/cm², and allowed to grow for 24 hrs. At this time (designated ‘0 hours’), the test agents were added to the cells, in 10% ethanol vehicle, such that the concentration of vehicle in the culture medium did not exceed 0.1%. The cells were then incubated for either 24 hrs or 72 hrs depending on the experiment. All treatment conditions were established in triplicate.

The level of mRNA for tgfb1, tgfb2 and tgfb3 were estimated by quantitative PCR. The media was removed and RNA was carefully prepared using the Ambion RNAqueous4PCR kit (Ambion#1914) in accordance with the manufacturer's instructions. The purity of the RNA and the quantity was assessed spectrophotometrically. RNA integrity was assessed by running a small aliquot of the samples (200 ng RNA) on the Agilent Bioanalyzer 2100 using the RNA 6000 Nano Assay. This analysis results in both a gel-like image as well as electrophoretic data. Indications of RNA degradation are: (a) decreasing ratio of ribosomal bands (b) additional peaks bellow the ribosomal bands (c) Decrease in overall RNA signal (d) shift towards shorter fragments. All the mRNA samples used in the experiments presented here passed this RNA quality control step. Next, the RNA was converted to cDNA using the ABI High-Capacity cDNA Archive Kit, in accordance with manufacturer's instructions. The qPCR assay was then set up as follow: all cDNA samples were diluted to 180u1 total volume using Molecular grade water. Samples were vortexed to ensure thorough mixing. cDNA was aliquoted into ABI 384 well plates using a multi-channel pipette, 4.5 μl of sample into each well. Samples were assayed in triplicate (that is, nine determinations in total were made for each condition, being three separate assays on each of three replicate culture wells). The assay mix and Universal Master Mix were prepared and aliquoted across the 384 well plates 5.5 μl in each well, using a single channel pipette. The final composition of each reaction (10 μl) being: Primer/Probe assay mix (×20)=0.5 μl; Universal Master Mix=5 μl; cDNA=4.5 μl. The plates were then heat sealed using Abgene clear seal strong plastic heat seals, and then cycled and scanned using the ABI 9700HT under the following conditions: 95° C. for 10 minutes to activate the AmpliTaq GOLD, then 40 cycles of: 95° C. for 15 sec (Denaturation), 60° C. for 60 sec (Anneal/Extension). Analysis was carried out using the ABI SDS 2.1 software.

Quantitative PCR was performed using several primer/probe sets. In each case, the primer pair was designed using methods well known in the art, enclosing a small amplicon of 10-12 bp to which the labelled probe sequence is complementary. We used both commercially available primer/probe sets (ABI Taqman pre-validated assays), using both available sets in the ABI database for tgfb1 and tgfb2, as well as the single available set for tgfb3. In addition, we used our own manual design probe/primer set for each of tgfb1, tgfb2 and tgfb3. For normalisation, we used TATA-binding protein (TBP) as the primary normalisation standard, with GAPDH as an alternative for confirmation, using ABI Taqman pre-validated probe/primer sets. The selection of normalisation control is essential: beta-actin is frequently used by others in such experiments but it is inappropriate because expression of all actin isotypes is modulated by TGF-beta, and agents which increase TGF-beta production will increase beta-actin mRNA (at least in some cell types) with a significant risk of a false negative outcome (since the elevated level of tgfb1 mRNA is ratio against an elevated level of beta-actin mRNA, yielding a ratio close to 1). Neither TBP nor GAPDH mRNA levels are affected by TGF-beta and are therefore equally suitable, however TBP was selected as the primary normalisation standard because the absolute level of mRNA in most cell types is similar to that of the TGF-beta isoforms, resulting in lower errors on calculating the ratio, and improving the power of the experiment.

In all quantitative PCR experiments, several additional controls were included: firstly, a control for each sample is run with no reverse transcription to ensure the mRNA sample is not contaminated with genomic DNA (although the manual design probe/primer sets were all selected to cross an exon/exon boundary, ensuring no detectable amplicon is produced from genomic DNA templates). Secondly, a control reaction is run using serial 2-fold dilutions of a standard cDNA preparation (prepared from commercially available IMAGE clones). This control ensures that the results for the unknown samples are obtained in the linear range of the amplification process, and are therefore truly quantitative. Failure of either control led to the repeat of the entire experiment. Typically, the half maximal signal was obtained after 20-30 amplification cycles.

For each condition, the cycle time to half maximal signal (Ct) was converted into relative amount of mRNA (Rt) using the following equation:

Rt=(2̂(K−Ct))/(2̂(K−Cn))

where K is a constant and Cn is the cycle time to half maximal signal for the primary normalisation standard TBP. The relative amount of mRNA for each TGF-beta isoform in cells treated with various agents is then present as a percentage of the relative amount of mRNA for the same isoform in cells treated with vehicle alone, using the standard deviation between triplicate wells to determine the statistical significance of the effect of the agent under study (using Student's unpaired t-test, with p<0.05 taken to indicate a statistically significant result).

TGF-beta1 protein levels in the medium was measured using the Quantikine ELISA kit (R&D Systems) in accordance with the manufacturer's instructions, except that the medium was pre-activated by addition of an equal volume of 2.7M HCl 10M Urea, and then neutralised by addition of the same volume of 1M HEPES 2M NaOH. This activation procedure (unlike the recommended procedure using 1M NCl) ensures that all TGF-beta1 containing complexes are fully activated, and is generally considered to represent a measure of ‘total’ TGF-beta1 (see, for example, the discussion in Grainger et al. Cytokine Growth Factor Rev. (2000) 11:133-45). Note that TGF-beta proteins are well known to interact with many different matrix proteins and cell surface proteins, including proteoglycans, low affinity type III receptors, fibronectin and collagen, and consequently the amount of TGF-beta protein in the medium may not represent the total amount synthesised by the cells, and a negative result in the assay for TGF-beta protein may represent a false negative.

Finally, TGF-beta activity was estimated by the direct effects on smooth muscle cell proliferation within the assay. For these experiments, the test agents were added, in triplicate, to cells both in the presence and absence of a neutralising antibody to TGF-beta (AB-100-NA; R&D Systems at 10 μg/ml, which is able to neutralise at least 25 ng/ml recombinant active TGF-beta1; R&D Systems). After 72 hrs incubation, the cells are washed and then released completely using 0.02% trypsin/EDTA (Gibco) at 37° C. for 10 mins, then counted manually using a haemocytometer. Importantly, the number of cells at 0 hrs must also be determined (using the same counting method, on a replicate set of three wells plated out specifically for the purpose). For each treatment condition, the TGF-beta-dependent inhibition of proliferation is expressed as the fold increase in the cumulative population doubling time in the absence of the neutralising antibody compared to the cumulative population doubling time in the presence of the neutralising antibody. As a control, an additional set of six wells are treated with 10 ng/ml recombinant active TGF-beta1 (R&D Systems) which results in a fold-increase in cumulative population doubling time of at least 1.5 fold. Similarly, the fold-increase in cumulative population doubling time in the presence of vehicle only must be less than 1.1-fold. Failure of either control led to the repeat of the experiment.

### Results

The effect of various concentrations of Tamoxifen (T) and aspirin (A), administered either separately or as a mixture according to the invention, on the mRNA levels of tgfb1, tgfb2 and tgfb3 after 24 hrs treatment was determined as described, and shown in Table 1.

Tamoxifen significantly increased tgfb1 mRNA and tgfb3 mRNA, but not tgfb2 mRNA at doses of 10 μM and above. At concentrations above 33 μM Tamoxifen was toxic to the cells (most likely due to the detergent properties of the molecule, with a critical micelle concentration of approximately 50 μM in the presence of 20% foetal calf serum). At 33 μM, TMX increased the level of tgfb1 mRNA by approximately 1.4 fold.

Aspirin had no statistically significant effect on the mRNA levels of any of the three isoforms of TGF-beta at any concentration tested (up to 100 μM).

Unexpectedly, administration of Tamoxifen and aspirin as a mixture showed a marked synergistic effect on TGF-beta mRNA levels. At all doses of aspirin tested, the concentration of Tamoxifen required to achieve a statistically significant increase in tgfb1 mRNA levels was markedly reduced. In addition, in the presence of aspirin the maximal effect of Tamoxifen on tgfb1 levels (at Tamoxifen concentrations above 10 μM) was almost double that achieved in the absence of aspirin.

TABLE 1
Level of mRNA, estimated by qPCR.

| | tgfb1 | tgfb2 | tgfb3 |
|---|---|---|---|
| 3.3 μM T | 0.95 ± 0.16 | 0.99 ± 0.12 | 0.92 ± 0.16 |
| 10 μM T | 1.27 ± 0.09 * | 1.05 ± 0.17 | 1.19 ± 0.21 |
| 33 μM T | 1.41 ± 0.08 * | 1.03 ± 0.06 | 1.31 ± 0.11 * |
| 10 μM A | 1.01 ± 0.04 | 0.92 ± 0.10 | 1.09 ± 0.08 |
| 33 μM A | 0.93 ± 0.11 | 1.00 ± 0.17 | 1.06 ± 0.13 |
| 100 μM A | 1.06 ± 0.08 | 1.03 ± 0.16 | 0.94 ± 0.09 |
| 10 μM A + 3.3 μM T | 1.13 ± 0.04 * | n.d. | 1.03 ± 0.15 |
| 10 μM A + 10 μM T | 1.36 ± 0.17 * | n.d. | 1.31 ± 0.10 * |
| 10 μM A + 33 μM T | 1.48 ± 0.08 * | n.d. | 1.45 ± 0.03 * |
| 33 μM A + 3.3 μM T | 1.38 ± 0.07 * † | n.d. | 1.19 ± 0.11 * † |
| 33 μM A + 10 μM T | 1.52 ± 0.26 * | n.d. | 1.28 ± 0.38 |
| 33 μM A + 33 μM T | 1.69 ± 0.10 * † | n.d. | 1.51 ± 0.15 |
| 100 μM A + 3.3 μM T | 1.47 ± 0.15 * † | n.d. | 1.27 ± 0.08 * † |
| 100 μM A + 10 μM T | 1.71 ± 0.12 * † | n.d. | 1.26 ± 0.20 * |
| 100 μM A + 33 μM T | 1.86 ± 0.19 * † | 1.07 ± 0.10 | 1.58 ± 0.08 * † |

Levels of mRNA normalised to TBP are reported as a fold-change compared to cells treated with vehicle alone. Values are the mean of triplicate wells, error bars are standard deviations.
* p < 0.05 versus vehicle only, 2-tailed Student's t-test assuming homoscedacity.
† p < 0.05 versus the same concentration of Tamoxifen in the absence of aspirin by Student's t-test.
N.d. = not determined.
Factorial ANOVA demonstrated a significant interaction between Tamoxifen (T) and Aspirin (A) for tgfb1 (p = 0.006) but not for tgfb3 (p = 0.09).

The effect of various concentrations of Tamoxifen and aspirin, administered either separately or as a mixture according to the invention, on the level of TGF-beta1 protein in medium conditioned for 24 hrs or 72 hrs on smooth muscle cells was determined as described.

Tamoxifen had no statistically significant effect on total TGF-beta1 protein levels in conditioned medium either at 24 hrs or 72 hrs at any dose tested up to 33 μM. Doses of TMX above 33 μM were toxic to the cells after 24 hrs, and doses above 10 μM were toxic to the cells after 72 hrs.

Aspirin has no statistically significant effect on the total TGF-beta1 protein levels in conditioned medium at either 24 hrs or 72 hrs at any dose tested (up to 100 μM).

In contrast, the use of a mixture of Tamoxifen (10 μM) and aspirin (100 μM) significantly increased the level of total TGF-beta1 protein in the conditioned medium at both 24 hrs and 72 hrs. Although the extent of the increase was only modest (+14%; p<0.05 at 24 hrs, +19%; p<0.05 at 72 hrs), this observation is consistent with the large and unexpected increase in tgfb1 mRNA production in response to the combined composition.

The effect of various concentrations of Tamoxifen and aspirin, administered either separately or as a mixture according to the invention, on TGF-beta activity was indirectly determined by measuring the proliferation of the cells in the presence and absence of a neutralising antibody as described, and shown in Table 2.

Tamoxifen reproducibly increased the cumulative population doubling (CPD) time of the cells at 10 μM (CPD in the absence of neutralising antibody=1.4 fold higher than CPD in the presence of neutralising antibody). This is consistent with a significant increase in TGF-beta activity. Lower doses of Tamoxifen had no statistically significant effect on CPD and hence TGF-beta activity.

Aspirin also reproducibly increased the cumulative population doubling (CPD) time of the cells at 100 μM (CPD in the absence of neutralising antibody=1.25 fold higher than CPD in the presence of neutralising antibody). This is consistent with a significant increase in TGF-beta activity, albeit smaller in magnitude than that observed with 10 μM Tamoxifen. Lower doses of aspirin had no statistically significant effect on CPD and hence TGF-beta activity.

Once again, the use of a mixture of Tamoxifen and aspirin showed significant and unexpected synergy. Tamoxifen statistically significantly increased TGF-beta activity at doses down to 1 μM in the presence of 100 μM aspirin (representing a 10-fold decrease in the concentration of Tamoxifen required to achieve a significant increase in TGF-beta activity, estimated through its effects on cell proliferation).

TABLE 2
Effect of Tamoxifen (T) and Aspirin (A) on TGF-beta activity,
measured by the effect on SMC proliferation.

| | 0 μM T | 1 μM T | 3.3 μM T | 10 μM T |
|---|---|---|---|---|
| 0 μM A | 1.13 ± 0.14 | 1.06 ± 0.18 | 1.17 ± 0.21 | 1.40 ± 0.16 * |
| 10 μM A | 1.05 ± 0.09 | 1.11 ± 0.12 | 1.28 ± 0.15 * | 1.36 ± 0.06 * |
| 33 μM A | 1.13 ± 0.17 | 1.15 ± 0.26 | 1.44 ± 0.17 * | 1.32 ± 0.19 * |
| 100 μM A | 1.25 ± 0.06 * | 1.48 ± 0.16 * | 1.34 ± 0.10 * | 1.51 ± 0.21 * |

Under each condition the ratio of the proliferation rate (CPD) in the presence and absence of a neutralising anti-TGF-beta antibody is given, as the mean ± SD for triplicate determinations.
* p < 0.05 comparing the CPD in the presence of the antibody with the CPD in the absence of the antibody, confirming the presence of significant TGF-beta activity. In this assay, 10 ng/ml recombinant active TGF-beta1 (R&D Systems) increases the CPD by approximately 1.6 fold.
Factorial ANOVA demonstrated a significant interaction between Tamoxifen (T) and Aspirin (A) (p = 0.016)

### Conclusions

Taken together, these experiments show that Tamoxifen and aspirin show unexpected synergistic effects, and that the combination is considerably more potent and powerful as TGF-beta Production Stimulator than either compound administered separately, and indeed more powerful and potent than could have been predicted from a simple additive combination of their effects.

### Example 2

### Treatment of apoE-Deficient Mice with Tamoxifen and Aspirin

In order to examine the impact of combining Tamoxifen and aspirin as a mixture on the pro-coagulant effects of Tamoxifen, as well as on the anti-atherogenic properties (mediated, at least in part, through the TGF-beta Production Stimulating activity of Tamoxifen), mice prone to develop vascular lipid lesions were treated with Tamoxifen, aspirin and a mixture of the two agents according to the invention.

ApoE-deficient mice were selected because they are the most commonly used model of atherogenesis in rodents. In addition, the effect of Tamoxifen in this model has previously been well characterised (Circulation. 1997 Mar. 18; 95(6):1542-8). The extent of vascular lipid lesion formation at the aortic root, marked by staining for neutral lipid accumulation, was used as an indicator of therapeutic efficacy, while levels of TGF-beta1 protein in the blood vessel wall, measured by quantitative immunfluoresence, was used to demonstrate in vivo TGF-beta Production Stimulation. Finally, the extent of fibrin(ogen) deposition into the blood vessel wall, again measured by quantitative immunofluorescence, was used as an indicator pro-coagulant status and hence the risk of thrombotic complications.

### Methods

Adult male apoE-deficient mice, back-crossed onto a C57B16 background for at least five generations, were randomised into groups of eight animals at 12 weeks of age, such that each group had similar average body weight. Each group was fed Tamoxifen (1 mg/kg/day) or aspirin (30 mg/kg/day) or both compounds simultaneously, compounded into standard mouse chow (SDS), or normal mouse chow only, as the control group. Note that the mice receiving both compounds received food pellets into which a homogenous mixture of the ingredients had been compounded, and not a mixture of food pellets each containing one of the two active ingredients. The amount of food eaten and the body weight of each mouse was monitored daily for the first week, and once per week thereafter. After 12 weeks of treatment, the mice were sacrificed by CO₂ asphyxiation, and heart & lung blocks were excised, embedded in Cryo-M-bed (Bright Instruments, UK) without fixation and snap frozen in liquid nitrogen.

Sections were then prepared from the aortic root according to the Paigen Strategy (a defined sequence of cutting sections which allow reproducible comparison between animals; see for example Grainger et al. (1995) Nature Med. 1:1067-72 and the references therein). 4 μm crysosections were cut using a motordriven cryotome (Bright Instruments) and collected onto poly-L-lysine coated glass microscope slides, then fixed in ice-cold acetone for 90 secs, allowed to air dry and stored at −20° C. until analysed.

The extent of vascular lipid lesion formation was assessed by Oil Red O staining, with a Fast Green counterstain, a standard histological procedure well known in the art. Briefly, five sections (selected according to the Paigen strategy) from each animal were passed through graded alcohol solutions, then stained in Oil Red O for 12 minutes, washed several times and then counterstained by dipping in Fast Green for 5-10 secs, and then dip washed in several changes of clean water. After air drying, an appropriate mountant was applied (simple glycerol-based moutants are most appropriate for this task), followed by a coverslip using acrylic varnish surrounding the coverslip to hold it in place. Slides were then analysed using a simple inverted microscope (no phase contrast; 10× objective; Olympus AX series), fitted with a digital camera to capture images of the entire aortic wall. The images were subsequently processed, blind to the treatment status of the animal, using Openlab image analysis software (Improvision, UK) running on an Apple Macintosh computer. On each image, the user delineated the external elastic lamina, internal elastic lamina and the luminal surface of the vessel wall, and the software reported the total intimal area, the intimal:medial area ratio, the total neutral lipid area and the intimal lipid area. The values from each image were summed (except for intimal:media area ratio, which were averaged), and then the values from each slide were summed (or averaged, as above) to yield a value for each parameter for each animal. The mean and standard error across the group of animals treated under the same conditions is then reported, using Student's unpaired t-test or ANOVA as appropriate to determine statistically significant differences between the groups.

The amount of TGF-beta1 protein present in the blood vessel wall was determined by quantitative immunofluoresence, as previously extensively described (see Mosedale et al. (1996) J. Histochem. Cytochem. 44:1043-50 for a comprehensive discussion of the key factors in designing a quantitative immunofluoresence experiment; note that all the recommendations therein were rigorously applied during the experiments presented here). Briefly, five slides were selected according to the Paigen Strategy from each animal, each with two neighbouring 4 μm sections on the slide, and the sections were enclosed with a water-resistant barrier (using a PAP pen; Agar Scientific, UK) such that the enclosed area was approximately equal on all slides. Non-specific antibody binding was then blocked using 3% IgG-free bovine serum albumin (BSA; Sigma Chemical Company) in phosphate-buffered saline (PBS) pH7.4 for 2 hours at room temperature. The blocking solution was then gently removed, and replaced with 50 μl of a primary antibody (chicken anti-TGF-beta1; AB-101-NA; R&D Systems) in blocking buffer (PBS+3% BSA) and incubated overnight at room temperature in sealed, humidified chambers. As a control, two of the five slides from each animal received only blocking buffer in place of primary antibody solution (see Mosedale et al. (1996) J. Histochem. Cytochem. 44:1043-50 for a full discussion of the appropriateness of a no-primary-antibody control in quantitative immunofluoresence). At the end of this incubation, every slide was washed 3×3 mins in PBS, taking care to gently remove each wash prior to the next addition. Note that for a large experiment (with, for example 6 groups of 8 animals), a total of 240 slides need to be washed; in order to maintain an accurate 3 min wash duration, slides were washed in random batches of 20 slides). Immediately after the final wash, 100 μl of secondary antibody (donkey anti-chicken IgG minimum cross reactivity, FITC labelled, 50 μg/ml in blocking buffer; Jackson Immunoresearch) was added to every slide (including those which received blocking buffer in place of primary antibody). The slides were then incubated for a further 6 hours at room temperature in humidified chambers in the dark. Next, the 3×3 min washing procedure was repeated, followed by a single rinse in MilliQ and then the slides were allowed to airdry in the dark. An appropriate mountant (e.g. Citifluor AF-1, which contains an anti-fadant) was then added, followed by a coverslip held in place using acrylic varnish around the edges. Slides were stored in the dark at −20° C. until analysed, for a maximum of 18 hours.

From each slide (including the controls which received no primary antibody), four images of the aortic wall were captured, using the same microscope and image analysis system as for the Oil Red O analysis, except that images were captured in dark field mode, using a mercury burner with a 10 nm bandpass filter to excite the fluoraphore at 435 nm, and a dichroic set with a 460 nm cut-off mirror and a 10 nm bandpass emission filter at 495 nm (Olympus NIBA filter set). The brightness, exposure time and all other system parameters were kept identical throughout the image capturing phase, and all images were captured by a single operator blind to the treatment status of the animals in a single session. A single image was captured with no slide on the microscope stage, and was digitally subtracted from all other images prior to further analysis. Note that the lamp brightness and exposure time were selected so that the control slides (which received no primary antibody) had a mean grey level in the region of interest of approximately 10% of full scale deflection, and the brightest staining on slides which received the primary antibody yielded a mean greay level of 60-80% of full scale deflection, with less than 10% of pixels in the region of interest exceeding 95% of full scale deflection. After image capture, the user manually delineated the external elastic lamina and the luminal surface, and the mean grey level in this region of interest was reported. For each animal, a single value was calculated as the mean grey level in the region of interest averaged for the three slides which received primary antibody minus the mean grey level in the region of interest averaged for the two slides which received no primary antibody. The mean and standard error (in arbitrary units) was then reported for each group of eight animals, and either Student's unpaired t-test or ANOVA was used to assess statistically significant differences between the groups as appropriate.

The level of fibrin or fibrinogen deposited into the vessel wall was also estimated by quantitative immunofluoresence, using a method identical to that for TGF-beta1, except that the primary antibody was sheep anti-fibrin(ogen) (Chemicon; 2 μg/ml) and the secondary antibody was donkey anti-sheep IgG minimum cross-reactivity, FITC labelled (Jackson Immunoresearch), as previously described (Thromb Res. 2001 Apr. 1; 102(1):71-80).

### Results

Tamoxifen (1 mg/kg/day p.o) reduced vascular lipid lesion area by 72%, consistent with its known activity as a TGF-beta Production Stimulator. In contrast, aspirin (30 mg/kg/day p.o) reduced lipid lesion area by only 6% and this change was not statistically significant. The changes in intimal lipid lesion area with both drugs were similar to the changes in total lipid lesion area (Table 3). However, the combination of Tamoxifen and aspirin administered as a mixture in accordance with the present invention reduced both total lipid lesion area and intimal lipid lesion area more significantly than Tamoxifen alone, demonstrating an unexpected synergy between the two agents. It is likely that this synergy in vivo is a direct consequence of the significant increase in TGF-beta Production Stimulation activity observed for the combination in vitro (see Example 1).

TABLE 3
Effect of Tamoxifen and Aspirin on vascular lipid lesion development in apoE mice.

| | Lipid lesion | Intimal lipid | Total intimal | Intimal:media |
|---|---|---|---|---|
| | area (μm²) | lesion area (μm²) | area (μm²) | area ratio |
| Control | 38,170 ± 6,120 | 32,941 ± 5,928 | 66,129 ± 9.428 | 62 ± 11% |
| TMX (1 mg/kg/day) | 10,794 ± 2,820 * | 9,393 ± 2,221 * | 64,428 ± 4,294 | 60 ± 8% |
| Aspirin | 35,860 ± 8,091 | 31,006 ± 7,176 | 65,209 ± 6,712 | 62 ± 9% |
| (30 mg/kg/day) | | | | |
| TMX + Aspirin | 4,066 ± 912 * † | 3,982 ± 968 * † | 60,057 ± 6,433 * | 58 ± 8% |

Various measures of vascular lipid lesion development are shown (mean ± SD; n = 8 per group).
* p < 0.05 versus control by either Student's t-test or Mann-Witney U-test depending on the normality of each dataset.
† p < 0.05 versus TMX alone by the same statistical test.

Interestingly, Tamoxifen did not affect total intimal area, or the intimal:medial area ratio (Table 3), consistent with previous observations with TGF-beta Production Stimulators (for example Circulation 1997 Mar. 18; 95(6):1542-8 or J Biol. Chem. 1996 Dec. 6; 271(49):31367-71). This likely reflects the major mechanism of action of TGF-beta, to change plaque composition towards a stable plaque phenotype with less lipid content, but greater matrix composition. Aspirin did not affect intimal area or intimal:medial area ratio, and the combination of the two agents had no effect on intimal:medial area ratio, although intimal area was statistically significantly reduced by 9% (Table 3), underlying the synergy on multiple beneficial measures that is observed when using the combination in accordance with the invention.

Tamoxifen increased the level of TGF-beta1 in the blood vessel wall (Table 4), while aspirin had no effect. Consistent with the in vitro data (see Example 1), the level of TGF-beta1 in the vessel wall was increased to a greater extent with the combination of Tamoxifen and aspirin than with Tamoxifen alone (even though, under these conditions, aspirin alone did not have any activity as a TGF-beta Production Stimulator).

TABLE 4
Effect of Tamoxifen and Aspirin on TGF-b levels and
intravascular fibrinogen deposition in apoE mice.

| | TGF-b1 staining | Fibrin(ogen) staining |
|---|---|---|
| | (AU) | (AU) |
| Control | 67 ± 6 | 123 ± 13 |
| TMX (1 mg/kg/day) | 94 ± 9 * | 151 ± 7 * |
| Aspirin | 65 ± 11 | 70 ± 15 * |
| (30 mg/kg/day) | | |
| TMX + Aspirin | 114 ± 5 * † | 84 ± 16 * † |

Various measures of vascular lipid lesion development are shown (mean ± SD; n = 8 per group).
* p < 0.05 versus control by Student's t-test.
† p < 0.05 versus TMX alone by Student's t-test.

Although Tamoxifen and aspirin had marked synergistic activity, increasing TGF-beta1 levels and hence reducing the severity of vascular lipid lesion formation and changing plaque composition in favour of plaque stability, nevertheless the two agents had opposite effects on fibrin(ogen) deposition into the vessel wall. Tamoxifen increased fibrin(ogen) deposition by 23% (Table 4) consistent with the known pro-coagulant effects of triphenylethylenes. Aspirin reduced fibrin(ogen) deposition by 43% (Table 4) consistent with the anti-platelet activity of the drug. Interestingly, when the combination of the agents was administered in accordance with the invention, the anti-coagulant activity of aspirin dominated over the pro-coagulant activity of Tamoxifen. As a result, fibrin(ogen) deposition into the blood vessel wall was reduced by 32% in animals administered the combination, and this was not different from animals receiving aspirin alone.

### Conclusions

Taken together, these experiments show that Tamoxifen and aspirin show unexpected synergistic effects, and that the combination is considerably more potent and powerful as TGF-beta Production Stimulator in vivo than either compound administered separately, and indeed more powerful and potent than could have been predicted from a simple additive combination of their effects. Similarly, Tamoxifen and aspirin have synergistic effects on a number of lipid lesion size and composition parameters.

In addition, these results demonstrate that co-administration of aspirin abolishes the pro-coagulant activity of Tamoxifen and that, unexpectedly, the anti-coagulant activity of aspirin is fully dominant over the pro-coagulant effect of Tamoxifen. As a result, co-administration of aspirin would be expected to reduce or abolish any side-effects of Tamoxifen use which result from its pro-coagulant activity.

### Example 3

### Treatment of apoE-Deficient Mice with Fish Oils and Aspirin

In order to demonstrate the generality of the synergy shown in combination between TGF-beta Production Stimulators and aspirin, we selected another TGF-beta Production Stimulator which shares no known mechanistic or structural similarity to Tamoxifen: a mixture of omega-3 fatty acids in fish oil. ApoE-deficient mice were treated with fish oil (100 mg/kg/day p.o), aspirin (30 mg/kg/day p.o), or a combination of the two administered as a mixture.

It will be evident that since dietary supplementation with fish oil is not reported to be associated with any significant side-effects (unlike Tamoxifen), that example 3 is not itself a method according to the present invention (the utility of which is to abolish, reduce or ameliorate the side-effects from administering a TGF-beta Production Stimulator). Nevertheless, example 3 illustrates the generality of the principles of the invention.

### Methods

Adult male apoE-deficient mice were treated, in groups of 8, with fish oil (Seven Seas, UK), aspirin or both agents simultaneously from 12 weeks of age until 24 weeks of age, exactly as described in Example 2.

Mice were sacrificed and heart & lung blocks prepared and processed exactly as described in Example 2. Sections were prepared in accordance with the Paigen Strategy, as in example 2. The extent of lipid lesion formation was assessed by Oil Red 0 staining with Fast Green counterstain, exactly as in example 2.

The levels of TGF-beta1 and fibrin(ogen) in the vessel wall were determined by quantitative immunofluoresence, exactly as described in example 2.

### Results

Both fish oil, rich in omega-3 fatty acids, (100 mg/kg/day p.o), and aspirin (30 mg/kg/day p.o) each had no statistically significant effect on any measure of vascular lipid lesion area or plaque size when administered separately (Table 5). However, the combination of fish oil and aspirin administered as a mixture in accordance with the present invention reduced both total lipid lesion area and intimal lipid lesion area by 27% and 33% respectively, demonstrating a similar synergy between aspirin and fish oil, as was observed between aspirin and Tamoxifen in Example 2. The combination of fish oil and aspirin had no effect on intimal area, or the intimal:medial area ratio.

TABLE 5
Effect of Fish Oil and Aspirin on vascular lipid lesion development in apoE mice.

| | Lipid lesion | Intimal lipid | Total intimal | Intimal:media |
|---|---|---|---|---|
| | area (μm2) | lesion area (μm2) | area (μm2) | area ratio |
| Control | 38,170 ± 6,120 | 32,941 ± 5,928 | 66,129 ± 9.428 | 62 ± 11% |
| Fish oils | 37,618 ± 9,409 | 31,164 ± 8,431 | 69,024 ± 10,461 | 65 ± 10% |
| (100 mg/kg/day) | | | | |
| Aspirin | 35,860 ± 8,091 | 31,006 ± 7,176 | 65,209 ± 6.712 | 62 ± 9% |
| (30 mg/kg/day) | | | | |
| Fish oils + Aspirin | 27,864 ± 6,113 * † | 22,058 ± 5,438 * † | 63,938 ± 7,305 | 62 ± 7% |

Various measures of vascular lipid lesion development are shown (mean ± SD; n = 8 per group).
* p < 0.05 versus control by either Student's t-test or Mann-Witney U-test depending on the normality of each dataset.
† p < 0.05 versus fish oils alone by the same statistical test.

Fish oil increased the level of TGF-beta1 in the blood vessel wall by 17% (Table 6), consistent with its status as a TGF-beta Production Stimulator, albeit considerably less potent and powerful than Tamoxifen, while aspirin had no effect. Unexpectedly, the level of TGF-beta1 in the vessel wall was increased to a greater extent with the combination of fish oil and aspirin than with fish oil alone (even though, under these conditions, aspirin alone did not have any activity as a TGF-beta Production Stimulator). Indeed, the combination of fish oil and aspirin was as effective a TGF-beta Production Stimulator (in the blood vessel wall) as Tamoxifen when used alone.

TABLE 6
Effect of Fish Oils and Aspirin on TGF-b levels and
intravascular fibrinogen deposition in apoE mice.

| | TGF-b1 staining | Fibrin(ogen) staining |
|---|---|---|
| | (AU) | (AU) |
| Control | 67 ± 6 | 123 ± 13 |
| Fish oils | 77 ± 4 * | 114 ± 17 |
| (100 mg/kg/day) | | |
| Aspirin | 65 ± 11 | 70 ± 15 * |
| (30 mg/kg/day) | | |
| Fish oil + Aspirin | 98 ± 8 * † | 68 ± 9 * † |

Various measures of vascular lipid lesion development are shown (mean ± SD; n = 8 per group).
* p < 0.05 versus control by Student's t-test.
† p < 0.05 versus TMX alone by Student's t-test.

Fish oil alone had no effect on fibrin(ogen) deposition into the blood vessel wall (consistent with our findings that pro-coagulant activity is associated specifically with TGF-beta Production Stimulators of the triphenylethylene structural family). Aspirin significantly reduced fibrin(ogen) deposition into the blood vessel wall, consistent with its known anti-platelet activity, irrespective of whether it was administered alone or in combination with fish oil.

### Conclusions

Aspirin shows unexpected synergistic activity with a TGF-beta Production Stimulator entirely unrelated to Tamoxifen. We conclude that combinations of TGF-beta Production Stimulators with aspirin have unexpected synergistic benefits, which are greater than could be predicted by simple addition of the effects of the two agents.

### Example 4

### Treatment of Murine Collagen-Induced Arthritis with Tamoxifen and Aspirin

Rheumatoid arthritis is another disease associated with the loss of normal adult tissue architecture, and as a result is known to be effectively treated by TGF-beta Production Stimulators. The collagen-induced arthritis model in mice has been widely used as an animal model of the human disease (Nature 1980; 283:666-668). In this model, mice are sensitised with a systemic injection of type II collagen in the presence of an immune system adjuvant (usually Complete Freund's Adjuvant (CFA)), and following a second exposure to the collagen antigen develop a severe arthralgia in all four limbs which progresses in severity of a three week period, and then spontaneously resolves. The thickness of the footpad, measured using an accurate micrometer, is a useful marker of both inflammation and local edema, which in turn are surrogate markers of joint damage and disease progression.

Mice developing collagen-induced arthritis were treated with either Tamoxifen alone, or a combination of Tamoxifen and aspirin as a mixture in accordance with the present invention.

### Methods

Collagen-induced arthritis was induced in adult male DBA/1 mice as previously described (Courtenay et al. (1980) Nature 283:666-8). Adult (12 week old) male DBA/1 mice were divided into groups of six animals. Mice received 100 μg per mouse of bovine type II collagen (Sigma) in Freunds Complete Adjuvant (FCA) by the subcutaneous route, while a control group received an identical injection of type I collagen in FCA. Twenty-one days later, all groups of mice received an intraperitoneal booster injection containing 100 μg per mouse of the same collagen type as previously, but in the absence of adjuvant. Various groups of mice then received either Tamoxifen (1 mg/kg/day p.o); Tamoxifen (1 mg/kg/day p.o) and aspirin (30 mg/kg/day p.o) as a mixture; or normal mouse chow (RM-1; SDS Ltd). Foodpad thickness was determined each day using a digital caliper EC1507 (Moore & Wright) accurate to 0.01 mm. The thickness of each footpad was determined three times on each day by a single operator blind to the treatment condition of the mouse, and the footpad thickness of each mouse on each day was reported as the mean for the three measurements for all four paws. Errors represent the standard error of the mean among the six animals in each group.

A direct ELISA for anti-collagen type II antibodies was used as an alternative assessment of the autoimmune response. Bovine type II collagen was coated onto Maxisorp 96-well ELISA plate wells (Nunc) at 1 μg/well in 50 μl of 50 mM sodium carbonate pH9.0 for 2 hours at 4° C. Wells were blocked with 5% Tween-20/5% sucrose in phosphate-buffered saline for 1 hour at room temperature. Mouse serum at various dilutions was incubated with the collagen-coated wells for 2 hours at room temperature. After three quick washes with tris-buffered saline +0.05% Tween-20, bound murine antibody was detected using a range of different anti-mouse IgG peroxidase conjugates (Pan-IgG A-9174 Sigma Chemical Co; IgA A-4789 Sigma Chemical Company; IgG1, G2a, G2b and G3 Southern Biotechnology) at 1:5000 dilution in tris-buffered saline +0.05% Tween-20 for 1 hour at room temperature. After three further washes, bound peroxidase label was visualised with K-BLUE chromogenic substrate (Skybio, Ltd.) for 20 minutes at room temperature, and the concentration of murine anti-collagen IgG in the serum quantitated by interpolating a standard curve constructed using known concentrations of the murine monoclonal anti-collagen type II IgG, CIICI (NIH Developmental Studies Hybridoma Bank).

### Results

Within 96 h following the booster injection (by day 25), significant footpad swelling was noted in the group which received type II collagen, but normal mouse chow (the ‘disease’ group). The swelling increased dramatically, such that by day 29 the mean footpad thickness of the mice in the disease group had increased by 25%±2% compared with baseline (p<0.0001; Wilcoxon signed-rank test). The thickness of all four footpads in all six mice in this group had increased significantly compared with baseline by day 31 of the study. Where footpad swelling was most severe (approximately double the footpad thickness at baseline, in one paw from each of four different mice) marked erythema was noted. In the disease group, footpad swelling remained constant for several days, then began to decline between days 33 and 39. By day 39, the mean footpad thickness had returned to baseline. In contrast, the mice in the control group who had received type I collagen injections showed only a very small increase in footpad thickness (<5%), maximal on day 30, but this change did not reach statistical significance at any time point.

The mice in the treatment groups received identical injections of type II collagen to the mice in the disease group, but there was a much less marked increase in footpad thickness. Treatment with Tamoxifen alone from the time of the booster injection onwards reduced the maximal collagen-II induced footpad swelling in DBA/1 mice by 36% (p<0.01; Mann-Whitney U-test). Treatment with Tamoxifen and aspirin as a mixture according to the present invention reduced maximal collagen-II induced footpad swelling by 62% (p<0.001; Mann-Withney U-test). Furthermore, ANOVA with Scheffe's post hoc tests, confirmed that the reduction in footpad swelling achieved with the combination of Tamoxifen and aspirin was significantly greater than with Tamoxifen alone.

There was no change in the weight of any of the mice in any of the groups throughout the period of the experiment. Furthermore, the mice receiving chow containing the treatment agents consumed the same quantity of food as the mice receiving only normal mouse chow.

The amount of IgG class immunoglobulin directed against bovine collagen-II present in serum at the end of the experiment (day 39) was also measured. Less than 1 μg/ml anti-collagen-II IgG was detected in the serum from all six of the mice in the control group who had not been exposed to collagen-II antigen. In contrast, high but very variable levels of anti-collagen-II IgG was detected in the serum from the mice in the disease group. Individual mice had levels of anti-collagen-II IgG varying from approximately 50 μg/ml to more than 5 mg/ml with a median level of 708 μg/ml. Serum from mice treated with Tamoxifen alone contained significantly less anti-collagen-II IgG than serum from mice in the disease group, despite identical exposure to the antigen (median level 181 μg/ml; p<0.05 Mann-Whitney U test). Serum from mice treated with Tamoxifen and aspirin combination showed an even more marked reduction (median level 52 μg/ml; p<0.05 Mann-Witney U test). Since bound antibody was detected using a F(ab′)2 fragment conjugated to peroxidase, these observations are unlikely to be due to interference by rheumatoid factors in the serum. We conclude that treatment with TMX from the time of the booster injection markedly inhibited or delayed the generation of the antibody response to the injected antigen, and that combination therapy with aspirin showed an unexpected synergistic effect with Tamoxifen in this model.

Using different detection antibodies in the direct ELISA assay the class distribution of the antibodies against bovine collagen II was investigated. There was a large amount of anti-collagen II IgG2b antibody in the serum from the disease group, with small but detectable amounts of IgG1, G2a and G3 sub-classes. Anti-collagen II IgA was not detected. In the group treated with Tamoxifen only there was a reduction in levels of all the IgG subclasses, although this was only statistically significant for IgG2b and G3, with no evidence of any alteration in the predominantly IgG2b profile of class switching. However low, but detectable, levels of anti-collagen II IgA were now found in the serum of mice treated with TMX. In the group treated with the combination of Tamoxifen and aspirin, the decrease in IgG2b and IgG3 levels as well as the increase in IgA levels were more marked, and the change in IgA levels was statistically significant. Since TGF-beta is responsible for directing class switching to IgA, the alterations in the isotype distribution of the (albeit smaller) pool of anti-collagen II antibodies can be used as a surrogate measure of the TGF-beta activity in vivo.

TABLE 7
Effect of TMX treatment (±aspirin) on antibody class switching.

| | | | Disease + TMX + |
|---|---|---|---|
| Isotype | Disease | Disease + TMX | aspirin |
| Pan IgG (μg/ml) | 708 | 181 | 52 |
| IgG1 | 0.177 | <0.05* | <0.05* |
| IgG2a | 0.196 | 0.081* | 0.053* |
| IgG2b | 2.957 | 0.482* | 0.181*† |
| IgG3 | 0.198 | 0.069* | <0.05* |
| IgA | <0.05 | 0.101* | 0.282*† |

In each case, immunoglobulin binding to bovine collagen II in a direct ELISA is shown. Pan-IgG was detected using F(ab′)2 fragment of sheep anti-mouse IgG and the median concentration in serum is shown for each group of mice. For IgG1, G2a, G2b, G3 and A, the median absorbance from the direct ELISA using neat serum is shown.
*p < 0.05 (Mann-Witney U test) versus disease (untreated);
†p < 0.05 (Mann-Witney U test) versus disease treated with Tamoxifen (TMX) only.

### Conclusions

Treatment with TMX from the time of the booster injection markedly inhibited or delayed the development of arthritis in this model, as well as the generation of the antibody response to the injected antigen. Combination therapy with aspirin showed an unexpected synergistic effect with Tamoxifen in this model. Since aspirin has no molecular actions expected to yield beneficial effects in this model, the synergistic benefit observed on multiple end-points is likely to have resulted from the same synergistic stimulation of TGF-beta production that was observed in vitro (see Example 1) and in vivo in a mouse model of atherogenesis (see Example 2). Indeed, the class-switching to IgA among the autoantibodies detected here provides the first direct evidence that elevated TGF-beta production in response to the various treatments was responsible, at least in large part, for the beneficial effects which were observed.

### Example 5

### Effects of Tamoxifen/Aspirin Co-Administration in Man

In order to examine the impact of combining Tamoxifen and aspirin as a mixture on the pro-coagulant effects of Tamoxifen, as well as on levels of TGF-beta activity relevant to immune system regulation, we treated men with coronary heart disease with Tamoxifen (20 mg once daily p.o) for three months in the presence or absence of co-administered aspirin (75 mg once daily p.o).

The pro-thrombin time (PTT) was taken as a measure of coagulant status of the individuals. Class switching of anti-carbohydrate antibodies (which is known to relate to coronary artery disease status; see Mosedale et al (2006) J. Immunol. Meth. 309:182-91) as well as reflect the functional cytokine profile of the individual (compare with Example 4) was selected as a marker of the TGF-beta Production Stimulation activity of the treatments, and hence as a surrogate of the efficacy of the composition.

### Methods

Men with angiographically defined coronary artery disease (at least 50% stenosis of one of the three major coronary arteries) were recruited to the study, and given either Tamoxifen alone (20 mg once daily per os) if their current medicinal regimen did not include aspirin, or else Tamoxifen (20 mg once daily per os) and aspirin (75 mg once daily per os). Patients continued with all other medications (statins, ACE inhibitors, beta blockers, calcium channel blockers, diuretics etc), but no patients were taking warfarin or tirofiban. Eight patients received Tamoxifen only, and twelve patients received Tamoxifen plus aspirin.

The coagulant status of the individuals was assessed using the pro-thrombin time (PTT, measured clinically; Hinchingbrooke Hospital, UK). PTT was measured twice, one week apart, prior to beginning the study, and then after 90 days treatment. In each case, each patient acted as their own control, and the impact of the treatment on PTT was assessed using a paired Student's t-test comparing the PTT at 90 days with the average of the two baseline determinations.

Blood samples were also taken at baseline, immediately prior to beginning the drug treatments, and also after 90 days. The blood was drawn from the antecubital fossa using 19-gauge butterfly needles, and dispensed into polypropylene tubes, where it was allowed to clot for between 2 and 3 hours at room temperature. The clot was released, and the sample spun (450×g; 4 mins), and the serum supernatant removed, aliquoted and stored frozen at −80° C. until analysed.

The level of anti-carbohydrate antibodies was measured by direct ELISA as previously described (Mosedale et al (2006) J. Immunol. Meth. 309:182-91). Briefly, Maxisop 96-well plates were coated with BSA-alpha-Gal (Glycorex) at 1 μg/well in 200 μl of Sodium Carbonate pH9.0 overnight at 4° C. After three quick washes with PBS, non-specific binding was blocked using 3% BSA in PBS at room temperature for 1 hour. After three further washes the serum was added at various dilutions ranging from neat to 1:100 dilution in PBS, then incubated for 2 hours at room temperature with shaking. The serum was then discarded, the plate washed 5 further times with PBS+0.1% Tween-20, and replicate strips (each containing a full range of dilutions of the same serum sample) were exposed to highly specific monoclonal antibodies to each isotype, including IgG1, IgG2, IgG3, IgG4, IgM, IgA, IgE and IgD, at 1 μg/ml in blocking buffer. After a further hour at room temperature with shaking, the antibody was discarded and the plate washed 3 further times with PBS/Tween, before being exposed to donkey anti-mouse IgG minimum cross reactivity, coupled to horseradish peroxidase (Jackson Immunoresearch), at 1 μg/ml in blocking buffer for a further 1 hour at room temperature with shaking. After a final 3 washes in PBS/Tween and single wash in PBS, 200 μl of K-blue chromogenic substrate was added to each well. After a carefully controlled period, accurate to 5 seconds, equal for all wells, the reaction was stopped by the addition of 50 μl 2M HCl and the colour read at 450 nM. Results are reported for each isotype as the mean titre (that is, the dilution of serum which yielded 50% of the maximal signal) as well as the mean signal at that titre (since the maximum absorbance in many cases was considerably below full scale deflection).

### Results

Treatment with Tamoxifen alone for 3 months resulted in a small, but statistically significant increase in pro-thrombin time (+17%; p<0.05 paired Student's t-test), consistent with the known pro-coagulant side-effects of triphenylethylene drugs in other contexts, and with the increased fibrin(ogen) deposition observed in mice (see Example 2). In contrast, treatment with both Tamoxifen and aspirin had no effect on pro-thrombin time (−3%; n.s.), demonstrating that administration of this combination effectively reduced or eliminated the side-effects associated with Tamoxifen use.

The anti-carbohydrate antibody profile was complex, consistent with previous reports, dominated by the IgD isotype as expected among men with severe heart disease. Following 3 months treatment with tamoxifen alone, the titre and absorbance of the anti-carbohydrate IgD was significant decreased (Table 8), while the titre of the IgG2 isotype and the absorbance of the IgA isotype were significantly increased. These changes are consistent with stimulation of TGF-beta production, as well as with an anti-atherogenic shift in the functional cytokine profile. Unexpectedly, the increased titre of IgG2 isotype was markedly greater among the individuals receiving both Tamoxifen and aspirin, and the absorbance of the IgG2 isotype was also increased. The changes in both titre and absorbance of the IgA isotype were also larger than in those receiving Tamoxifen alone, although this difference did not achieve statistical significance in the present pilot experiment. The titre of the IgM isotype was also significantly decreased among those receiving Tamoxifen and aspirin, but not among those receiving Tamoxifen alone.

TABLE 8
Changes in anti-carbohydrate antibody profiles among
men with heart disease treated with Tamoxifen only
(20 mg once daily) or Tamoxifen (20 mg once daily)
and aspirin (75 mg once daily) for 3 months.

| | Tamoxifen only | Tamoxifen plus aspirin |
|---|---|---|
| IgG2 Absorbance | +0.61 ± 0.17 * | +0.94 ± 0.23 * † |
| IgG2 titre | +176% ± 43% * | +489% ± 98% * † |
| IgD Absorbance | −0.82 ± 0.51 * | −1.03 ± 0.68 * |
| IgD titre | −234% ± 113% * | −278% ± 124% * |
| IgA Absorbance | 0.31 ± 0.17 * | 0.59 ± 0.32 * |
| IgA titre | +14% ± 52% | +49% ± 68% |
| IgM Absorbance | −0.29 ± 0.36 | −0.17 ± 0.28 |
| IgM titre | +16% ± 28% | −42% ± 13% * † |

Changes in absorbance and titre are calculated versus baseline, reported as mean ± SD. Absorbance changes are for neat serum. Titre was calculated at the dilution of serum required to achieve a half-maximal absorbance (note that maximal absorbance may not have occurred at the highest serum concentration because of competing antibodies binding to limited solid-phase antigen - for a full discussion of the interpretation of titres in these experiments see J. Immunol. Meth. 309: 182-91).
* p < 0.05 versus baseline using Student's paired t-tes.
† p < 0.05 versus Tamoxifen only, using Student's unpaired t-test.

### Conclusions

These results demonstrate that in man, as in mice (see Example 2), the combination of aspirin and tamoxifen is a significantly more powerful and potent TGF-beta Production Stimulator than Tamoxifen alone, in this case marked by the alteration in the functional cytokine profile reflected in the isotype switching pattern of the natural anti-carbohydrate antibody pool. Furthermore, the co-administration of aspirin with Tamoxifen abolished the pro-coagulant side-effects of the triphenylethylene, consistent with the dominant effect of the anti-platelet activity of aspirin observed in the animal studies (Example 2).

The combination of Tamoxifen and aspirin is therefore of considerably greater utility in the treatment of heart disease, a disorder associated with the loss of normal adult tissue architecture, and therefore known to be amenable to treatment using TGF-beta Production Stimulators, than could have been predicted by the simple additive assessment of the effects of the compounds when administered separately. It was not previously known whether the pro-coagulant effects of Tamoxifen would be dominant over the anti-coagulant effects of aspirin, or vice versa. It was not previously known whether Tamoxifen and aspirin would show synergistic activity as a TGF-beta Production Stimulator. Since we have observed both advantages in mice and now in humans, we conclude that the combination has a significant advantage of the use of either medication separately.

### Example 6

### Effects of Tamoxifen/Clopidogrel Co-Administration in Man

In order to demonstrate the generality of combining Tamoxifen with anticoagulant medicaments to reduce or abolish the associated pro-coagulant side effects, we treated men with coronary heart disease with Tamoxifen (20 mg once daily p.o) for three months in the presence or absence of co-administered clopidogrel (75 mg once daily p.o), an anti-platelet agent structurally unrelated to aspirin, with a different molecular mechanism of action.

As in Example 5, the pro-thrombin time (PTT) was taken as a measure of coagulant status of the individuals and class switching of anti-carbohydrate antibodies was selected as a marker of the TGF-beta Production Stimulation activity of the treatments, and hence as a surrogate of the efficacy of the composition.

### Methods

Men with angiographically defined coronary artery disease (at least 50% stenosis of one of the three major coronary arteries) were recruited to the study, and given either Tamoxifen alone (20 mg once daily per os) if their current medicinal regimen did not include clopidogrel, or else Tamoxifen (20 mg once daily per os) and clopidogrel (75 mg once daily per os). Patients continued with all other medications (statins, ACE inhibitors, beta blockers, calcium channel blockers, diuretics etc), but no patients were taking warfarin or tirofiban. Seven patients received Tamoxifen only, and eight patients received Tamoxifen plus clopidogrel.

The coagulant status of the individuals was assessed using the pro-thrombin time (PTT, measured clinically; Hinchingbrooke Hospital, UK). PTT was measured twice, one week apart, prior to beginning the study, and then after 90 days treatment. In each case, each patient acted as their own control, and the impact of the treatment on PTT was assessed using a paired Student's t-test comparing the PTT at 90 days with the average of the two baseline determinations.

Blood samples were also taken at baseline, immediately prior to beginning the drug treatments, and also after 90 days, and serum prepared exactly as in Example 5. The level of anti-carbohydrate antibodies was then measured by direct ELISA using the procedure given in Example 5.

### Results

As in Example 5, treatment with Tamoxifen alone for 3 months resulted in a small, but statistically significant increase in pro-thrombin time (+19%; p<0.05 paired Student's t-test). In contrast, treatment with both Tamoxifen and clopidogrel had no effect on pro-thrombin time (−1%; n.s.), demonstrating that administration of this combination effectively reduced or eliminated the side-effects associated with Tamoxifen use, as previously observed for a combination of Tamoxifen and aspirin.

In contrast to the combination between Tamoxifen and aspirin, however, the combination of clopidogrel with Tamoxifen did not modulate the impact of Tamoxifen on the isotype profile of the anti-carbohydrate antibodies. The titre of IgD class anti-carbohydrate antibodies was statistically significantly reduced in both treatment groups, while the titre of the IgG2 class and the absorbance of the IgA class were both statistically significantly increased to a similar degree in both treatment groups.

### Conclusions

The combination of clopidogrel with Tamoxifen abolished the pro-coagulant side-effects of the triphenylethylene.

The combination of Tamoxifen and clopidogrel is therefore of considerably greater utility in the treatment of heart disease than the use of Tamoxifen alone because of the reduced pro-coagulant side-effects.

### DEFINITIONS

The term “about” refers to an interval around the considered value. As used in this patent application, “about X” means an interval from X minus 10% of X to X plus 10% of X, and preferably an interval from X minus 5% of X to X plus 5% of X.

The use of a numerical range in this description is intended unambiguously to include within the scope of the invention all individual integers within the range and all the combinations of upper and lower limit numbers within the broadest scope of the given range. Hence, for example, the range of 1 to 6 carbon atoms specified in respect of (inter alia) formula I is intended to include all integers between 1 and 6 and all sub-ranges of each combination of upper and lower numbers, whether exemplified explicitly or not.

As used herein, the term “comprising” is to be read as meaning a fixed dose combination of the agents which are stated comprise the composition of the invention, such that the components are mixed together as part of the manufacturing process, forming an essentially homogenous mixture. For the avoidance of doubt, the co-administration of the two agents which comprise the composition of the invention, even if simultaneous, would not constitute a “mixture” as defined herein. However, as noted above, chemical combinations of the components which comprise the mixture (such as a salt) is envisaged, and constitutes a mixture (or two components in a mixture of three or more components) in accordance with this definition.

As used herein, the term “TGF-beta Production Stimulator” is used to describe an agent which increases cellular production of the cytokine, TGF-beta. Methods to determine whether an agent is a TGF-beta Production Stimulator are well known in the art (see for example U.S. Pat. No. 6,410,587 which is incorporated by reference herein). For example, cultured cells may be exposed to the candidate agent in vitro, and after a period of time the amount of TGF-beta protein or mRNA is assessed by methods well known in the art (such as quantitative PCR or ELISA). In the event that the amount of TGF-beta mRNA or protein is greater in the cells treated with the candidate agent compared to cells treated with any vehicle alone, and that such difference is statistically significant assessed by methods well known in the art (such as Student's t-test), then the agent has been proven to be a TGF-beta Production Stimulator. Alternatively, an animal may be exposed to the candidate agent in vivo, and after a period of time the amount of TGF-beta protein or mRNA is assessed in various target tissues, using methods well known in the art (including quantitative PCR, immunohistochemistry and ELISA). In the event that the amount of TGF-beta mRNA or protein is greater in one or more tissues from the animals treated with the candidate agent, compared to the same tissue from animals treated with any vehicle alone, and that such difference is statistically significant, then the agent has been proven to be a TGF-beta Production Stimulator. Note that multiple valid tests for defining a TGF-beta Production Stimulator have been described in the art, and that various factors can result in a false negative result in one or more tests: consequently, a negative result in one such test does not preclude the possibility that the agent is a TGF-beta Production Stimulator. As a result, a valid and reproducible demonstration that an agent increases TGF-beta in one test is by itself sufficient to prove conclusively that the candidate agent is a TGF-beta Production Stimulator.

As used herein, the term “TGF-beta” is used to mean any of the mammalian isoforms of TGF-beta e.g. TGF-beta1, TGF-beta2 and TGF-beta3, as well as their heterodimeric products, TGF-beta1.2, TGF-beta 1.3 and TGF-beta2.3.

As used herein, the term “aspirinate” is used to designate a general class of aspirin-like compounds containing a carboxylate group able to form a salt, which includes those members of the class of compounds of structure III that are able to form carboxylate salts, together with salts of aspirin (acetylsalicylic acid) and salicylic acid. As used herein, the term aspirinate designates the said compound in its salt form (that is, sodium acetylsalicylate is an aspirinate according to this designation, but acetyl salicylic acid itself is not). The positively-charged counterion of the aspirinate may include, but is not limited to, sodium, potassium, copper or positively charged ions derived from organic bases (such as Tamoxifen).

Unless otherwise defined, all the technical and scientific terms used here have the same meaning as that usually understood by an ordinary specialist in the field to which this invention belongs. Similarly, all the publications, patent applications, all the patents and all other references mentioned here are incorporated by way of reference (where legally permissible).

1. A pharmaceutical composition comprising a mixture of first and second active
ingredients,
wherein the first active ingredient is tamoxifen aspirinate and the second active ingredient is clopidogrel.

2. The pharmaceutical composition according to claim 1, wherein the mixture is an essentially homogeneous mixture.

3. The pharmaceutical composition according to claim 1, wherein the first and second active ingredients, together with any excipients and/or carriers, are formulated as a single tablet.

4. The pharmaceutical composition according to claim 3, wherein the dose of tamoxifen is 15 mg per tablet.

5. The pharmaceutical composition according to claim 1 wherein the dose of clopidogrel is between two and four times the dose of tamoxifen aspirinate.

6. The composition of claim 1, in a tablet, capsule, or gel form.

7. The composition of claim 1 in a solid form selected from: powder, granule, tablet, gelatin capsule, liposome, and suppository.

8. The composition of claim 1, further comprising a solid support selected from Calcium Phosphate, magnesium stearate, talc, a sugar, starch, gelatin, a cellulose, polyvinylpyrrolidine, and wax.

9. The composition of claim 8, wherein the solid support is a sugar, and is lactose or dextrin.

10. The composition of claim 8, wherein the solid support is a cellulose selected from methyl cellulose, and sodium carboxymethyl cellulose.

11. The composition of claim 1, in a liquid form selected from: a solution, an emulsion, a suspension, and a syrup.

12. The pharmaceutical composition according to claim 1 further comprising a third active ingredient in an amount selected to reduce the side effects associated with tamoxifen administration.

13. The pharmaceutical composition according to claim 12 wherein the third active ingredient is aspirin.
